(19) United States
(12) Patent Application Publication
(10) Pub. No.: US 2008/0183125 A1
(43) Pub. Date: Jul. 31, 2008

(54) Ear cleaning apparatus

(76) Inventor: Anthony F. Issa, Orlando, FL (US)

Correspondence Address: William M. Hobby, III;#375, 157 E. New England Avenue, Winter Park, FL, 32789, US

(21) Appl. No.: 11/698,933

(22) Filed: Jan. 29, 2007

(51) Int. Cl. A61F 11/00; A61F 7/12

(52) U.S. Cl. 604/26; 606/28; 606/162; 607/113

(57) ABSTRACT

An ear cleaning apparatus for removing ear wax from an ear canal has a housing having an ear insertion tip shaped to fit into a person's ear. The housing has first and second passageways with each passageway having an opening in the insertion tip adjacent to each other. The first passageway has an air bleed thereinto. A heater is mounted in the housing first passageway for generating heat, such as with a butane burner. A fan is mounted in the second passageway for drawing air thereinto through the second passageway opening from the first passageway opening when the ear insertion tip is inserted into an ear thereby drawing heated air from the first passageway into the ear and into the second passageway.

### BACKGROUND OF THE INVENTION

The present invention relates to an apparatus for removing fluid, ear wax and debris from the ear canal, and especially to an ear wax remover having a constant flow of heated air to vaporize and remove the ear wax.

In the past, there have been numerous devices for extracting fluids as well as particulate matter from different orifices and tissues of human and animal bodies. These devices mostly have specialized application and are not generally suitable for removing ear wax and fluids from the human ear. The ear canal is prone to the accumulation of ear wax and to the collection of water after swimming while in the water. A non-mechanical handheld curettes have been the traditional method of wax removal by physicians. Fluid removal is typically performed in a physicians's office by using large electrical powered vacuum pumps connected to tubing with narrow gauge tips and upon the direct visualization to evacuate fluid and debris. Cotton tipped swabs are often used inappropriately in the ear canal to remove wax and fluid but can cause trauma to the tissue of the ear as well as worsen the impaction of fluid and debris.

The present invention enables a person without medical training to safely remove ear wax as well as other fluids and debris from the ear canal without causing trauma to the ear drum or skin of the ear canal. The present invention is portable, lightweight and inexpensive to manufacture, and highly efficient at removing ear wax by first softening the ear wax with heated air and removing the softened ear wax with a vacuum.

Prior art U.S. patents for removing fluids from the ear can be seen in the Spilman U.S. Pat. No. 6,059,803 for a portable handheld ear vacuum device for removing fluid and debris from the ear canal which is essentially a fan drawing a vacuum from a nozzle that fits within the ear. The Wang U.S. Pat. No. 6,406,484 is an apparatus for use in the removal of impacted cerumen from the auditory canal and is a handheld instrument have an auditory canal plug that seals over the auditory canal and has a flow of air through the auditory canal plug and into the vacuum chamber. The Hakky et al. U.S. Pat. No. 6,023,639 is a non-invasive bodily fluid withdrawal and monitoring system for withdrawing and monitoring bodily fluids from a person's skin. The Ginsberg U.S. Pat. No. 5,309,899 is an automated ear cleansing device for cleansing a patient's ear where a liquid is heated and applied through a nozzle to the ear canal to flush the ear canal with the heated liquid. The Eichenlaub U.S. Pat. No. 4,793,352 is a heat transfer device which utilizes a vaporization unit wherein heat transfer material is vaporized. It can be used for removing excess ear wax. The Ginsberg U.S. Pat. Nos. 5,309,899 and 5,527,275 are each directed towards an automatic ear cleaning device which applies the heated liquid under pressure and varies the flow rate of the liquid through a nozzle. The Wang U.S. Pat. No. 6,991,638 is an ear vacuum for cleaning a person's ears and has a fan to provide a vacuum for removing debris from the ear. The Wim U.S. Pat. No. 6,187,021 is an ear cleaning portable rotary device having a rotary brush for inserting into the ear canal.

The present invention provides an ear cleaning apparatus which removes ear wax from an ear canal by creating a flow of heated air into the ear canal and through the ear canal and back into the collection chamber in which the heated air softens the ear wax which is withdrawn from the ear into a collection chamber.

### BRIEF DESCRIPTION OF THE DRAWINGS

Other objects, features, and advantages of the present invention will be apparent from the written description and the drawings in which:

FIG. 1 is a perspective view of an ear cleaning apparatus being inserted into a person's ear; and

FIG. 2 is a sectional view of an ear cleaning apparatus and a person's ear canal showing ear wax being removed from the person's ear.

### SUMMARY OF THE INVENTION

An ear cleaning apparatus for removing ear wax from an ear canal has a housing having an ear insertion tip shaped to fit into a person's ear. The housing has first and second passageways with each passageway having an opening in the insertion tip adjacent to each other. The first passageway has an air bleed opening thereinto. A heater is mounted in the housing first passageway for generating heat, such as with a butane burner. A fan is mounted in the second passageway for drawing air thereinto through the second passageway opening from the first passageway opening when the ear insertion tip is inserted into an ear thereby drawing heated air from the first passageway into the ear and into the second passageway. A collection chamber is located in the second passageway for collecting heated ear wax or the like therein. The heater has an igniter having a switch mounted onto the housing and has a battery mounted in the housing for powering the fan. A collection chamber has an exhaust port with a filter cover.

### DESCRIPTION OF THE PREFERRED EMBODIMENT

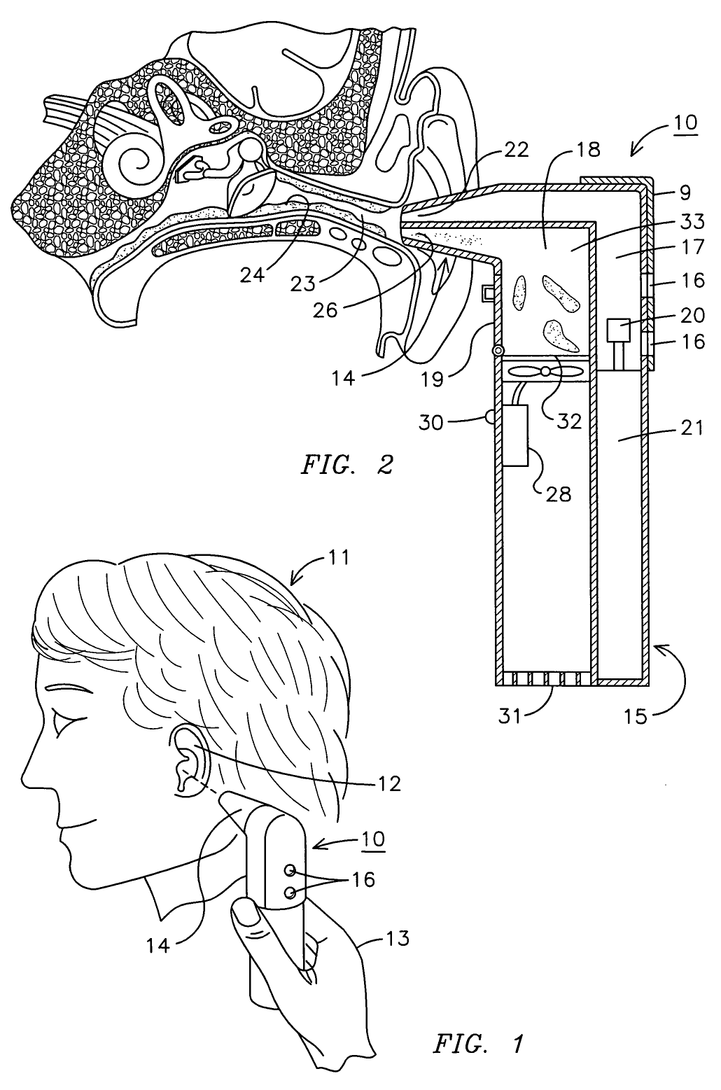

Referring to FIGS. 1 and 2 of the drawings, an ear piece cleaning apparatus 10 is show in FIG. 1 next to a person's 11 ear 12 and held by the person's hand 13. The ear cleaning apparatus 10 has an ear insertion tip 14 being positioned in FIG. 1 for placing in the person's 11 ear. The ear cleaning apparatus 10 has a casing 15 having a pair of openings 16 therein.

As seen in FIG. 2, the casing 15 has a passageway 17 and a separate passageway 18 therein. The passageway 17 has a butane heater 20 therein connected to a butane tank 21. The passageway 17 terminates in the tip 14 with an opening 22. The tip 14 is inserted in the person's ear 12 and into the ear canal 23 which is shown having accumulated ear wax 24 therein and having the ear drum 25 at the end of the opening of the ear canal 24. The housing includes a heat shield 9 shielding the burner 20.

The second passageway 18 terminates in the tip 14 with an opening 26 which is directly adjacent to the opening 22 from the passageway 17 placing the openings to the two passageways adjacent to each other. The openings 22 and 26 being adjacent each other at the end of the tip 14 allows for the continuous flow of air flow one to the other. The passageway 18 has an electric fan 27 mounted therein connected to a battery 28.

A switch 30 operates the fan 27. Fan 27 directs air towards an air exhaust 31 and has a filter 32 thereover to form a collection chamber 33 in the passageway 18. A hinged door 19 allows entry into chamber 33 to clean out the chamber of accumulated ear wax. The switch 30 also simultaneously, with turning on the fan, ignites the butane burner element 20 to produce heat at the same time the fan 27 is drawing air through the passageway 18 into the collection chamber 33 and out the air exhaust 31. When the ear cleaning apparatus 20 is inserted in the ear 12 into the outer part of the ear canal 23, both tip openings 22 and 26, positioned adjacent each other are positioned into the ear canal so that when the fan 27 is drawing air through the opening 26, the air is being pulled through the opening 22 of passageway 17 and through the openings 16 adjacent the heater 20. Thus, the air being drawn into the passageway 17 is being heated by the heating element 20 and is being drawn into the ear canal by the air being pulled from the ear canal into the opening 26 and passageway 18 by the fan 27. The heated air entering the ear canal will soften and vaporize the ear wax 24 allowing it to be drawn out by the air flow generated by the fan 27. The ear wax is collected in the collection chamber 33 and is blocked from being exhausted from the collection chamber by the filter 32.

A continuous path of air is drawn through the opening 26 past the heating element 20 through the passageway 17 and opening 22 into the ear canal and then into the opening 26 and passageway 18 by the blower 27. A continuous passageway is formed since the tip 14 is sealed against the ear canal 23.

It should be clear at this time that an ear cleaning apparatus has been provided which is a handheld portable device which can be used by an individual without medical supervision and in which a continuous passageway is provided for air entering the device passing through the ear canal and out an exhaust. The ear wax is vaporized in the canal and passes into a collection chamber. However, the present invention should not be construed as limited to the forms shown which are to be considered illustrative rather than restrictive.

I claim:

1. An ear cleaning apparatus for removing ear wax from an ear canal comprising:
a housing having an ear insertion tip shaped to fit into a person's ear, said housing having first and second passageways, each passageway having an opening in said insertion tip adjacent to each other, said first passageway having an air bleed thereinto;
a heater mounted in said housing first passageway for generating heat therein;
a fan mounted in second passageway for drawing a fluid thereinto through said second passageway opening and from said first passageway opening when said ear insertion tip is inserted into an ear, thereby drawing heated air from said first passageway into said ear and into said second passageway;
a collection chamber located in said other passageway for collecting fluids therein;
whereby said ear cleaning apparatus can heat and remove ear wax.

2. The ear cleaning apparatus in accordance with claim 1 in which said heater is a butane heater having an igniter.

3. The ear cleaning apparatus in accordance with claim 2 in which said heater igniter has a switch mounted on said housing.

4. The ear cleaning apparatus in accordance with claim 3 in which said first passageway air bleed includes a plurality of air bleeds.

5. The ear cleaning apparatus in accordance with claim 3 including a electric power source for operating said fan.

6. The ear cleaning apparatus in accordance with claim 5 in which said power source includes a battery.

7. The ear cleaning apparatus in accordance with claim 3 in which said collection chamber has an exhaust port.

8. The ear cleaning apparatus in accordance with claim 7 in which said collection chamber exhaust port has a filter cover.
